(19) United States
(12) Patent Application Publication
(10) Pub. No.: US 2012/0150099 A1
(43) Pub. Date: Jun. 14, 2012

(54) IONTOPHORETIC TRANSDERMAL DEVICE

(76) Inventor: TIMOTHY WEI-HANG, TAOYUAN COUNTY (TW)

(21) Appl. No.: 13/316,879

(22) Filed: Dec. 12, 2011

Related U.S. Application Data
(60) Provisional application No. 61/422,164, filed on Dec. 12, 2010.

(51) Int. Cl. A61N 1/30

(52) U.S. Cl. 604/20

(57) ABSTRACT

An iontophoretic transdermal device includes an applicator, an electric pathway and at least one electrode. The applicator has a first surface and the electric pathway is on the first surface. The electrode is also disposed in the first surface. The electric pathway is electrically connected with the electrode in order to carry the electric current into the electrode. Moreover, the electrode protrudes above the first surface in order to have a direct or indirect contact with the covered skin area substantially without gap.

### RELATED APPLICATION

Priority of US Provisional Patent Application Ser. No. 61/422,164, filed Dec. 12, 2010, incorporated herein by reference, is hereby claimed.

### FIELD OF INVENTION

The present invention generally relates to the filed of iontophoresis, and, more particularly, to inducing the agents into the skin with high permeability under the influence of an iontophoretic device and conformed cover to the contact skin.

### RELATED ART

Iontophoresis employs an electromotive force and/or current to transfer an active agent such as an ionic drug or other therapeutic agent to a biological interface, for example, skin or mucus membrane.

Iontophoretic devices typically include an active electrode assembly (usually an anode) and a counter electrode assembly, each coupled to an opposite pole or terminal of a voltage source, such as a chemical battery or an external power station connected to the iontophoretic devices via electrical leads. Each electrode assembly typically includes a respective electrode element to apply an electromotive force and/or current.

The active agent may be either cationic or anionic, and the voltage source can be configured to apply the appropriate voltage polarity based on the polarity of the active agent. Iontophoresis may be advantageously used to enhance or control the delivery rate of the active agent. The active agent may be stored in a reservoir such as a cavity. Alternatively, the active agent may be stored in a reservoir such as a porous structure or a gel.

One type of frequently used iontophoresis device is an iontophoretic patch, which includes an electrical power supply, and usually two separate patches with an anode on one piece and a cathode on the other, wherein both of the electrodes are electrically connected to the electrical power supply. The patches may have a chamber to contain the active agent and attach to the skin. This kind of iontophoresis device can perform a transdermal electrokinetic mass transformation to induce the agents into the skin. However, due to the construction of the pieces, such a design may be limited to application to smooth skin areas like the thigh or back, while being inapplicable to more featured areas such as face. In addition, the active agent which is contacted with the cathode and anode must be separated and electrically insulated from each other to prevent the electrical circuit from short-circuiting which would interfere with the iontophoretic process.

Another kind of iontophoresis device is an iontophoretic stick with electrodes and a battery operated controller, which requires the user to hold the iontophoretic stick to contact the required contact point. The limitations of such stick are inconvenience during application and having only one contact point rather than a broad skin area. Furthermore, the electrodes of the iontophoretic stick must contact the skin with minimum gap, or the distribution of the electrical current may be uneven and cause skin burns.

### SUMMARY OF THE INVENTION

The present invention is generally directed to an iontophoretic transdermal device, which includes an applicator with a first surface. The applicator is shaped as an orthomorphic projection of the covered skin area and the first surface is configured to be toward the covered skin area. The first surface contacts the covered skin area directly, or contacts the covered skin area with the iontophoretic transdermal active agent in between. The transdermal device further includes an electric pathway which is on in the first surface of the applicator and configured to carry the electric current. At least one electrode is also disposed on the first surface and electrically connected to the electric pathway. To assure a better contact between the electrode and the covered skin area, the electrode is designed to protrude above the first surface such that the electrode is able to contact the covered skin substantially without a gap when the applicator is applied on the covered skin area.

The present invention also discloses a method of conducting an iontophoresis process. The process includes applying an iontophoresis active agent on the treated skin surface and placing a first surface of an applicator on the treated skin surface. The process further has a step of contacting the skin surface with at least one electrode, wherein the electrode is electrically connected to an electric pathway and the electric pathway is electrically connected to a first terminal, connecting the first terminal to a first pole of a voltage source. A step of applying a second terminal on a skin area different from the treated skin surface wherein the second terminal is electrically connected to a second pole of the voltage source, and another step of introducing DC current from the voltage source into the electric pathway and the electrode are further included. The process also has a step of inducing the active agent underneath the treated skin surface.

### BRIEF DESCRIPTION OF THE DRAWINGS

FIG. 1 is a perspective view illustrating one embodiment of the present disclosure.

FIG. 2 illustrates an embodiment of the present disclosure.

FIG. 3 illustrates a cross-sectional view of an embodiment of the present disclosure.

FIG. 4 illustrates a cross-sectional view of an embodiment of the present disclosure.

FIG. 5 illustrates an embodiment of the present disclosure.

FIG. 6 illustrates one embodiment of the present disclosure.

FIG. 7 illustrates one embodiment of the present disclosure.

FIG. 8 illustrates one embodiment of the present disclosure.

FIG. 9 illustrates one embodiment of the present disclosure.

### DETAIL DESCRIPTION OF THE INVENTION

An iontophoretic transdermal device is developed to induce the agent into the skin with high permeability and uniformity. The term agent used in this disclosure means any therapeutic or prophylactic agent that is suitable for transdermal administration and that can be used with the device described herein. The agent may also include a buffer solvent to comfort the treated skin or to reinforce the efficiency of the iontophoretic process, wherein such agent can be daubed over the treated skin area or applied on a patch which is disposed on the contact skin.

The invention is described by way of concrete embodiments illustrated in the accompanying drawings, but it should be noted that the invention is in no way limited thereto.

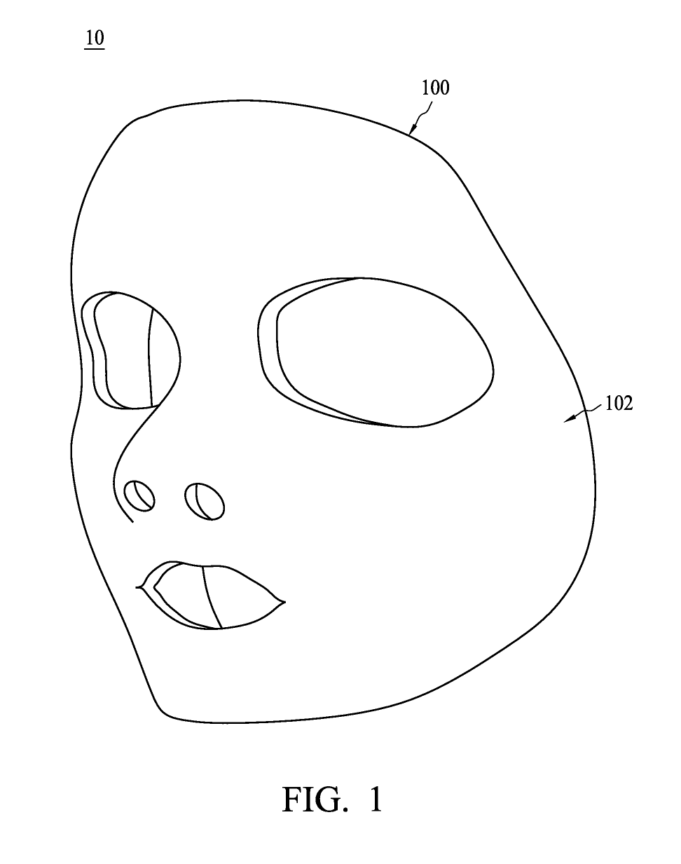

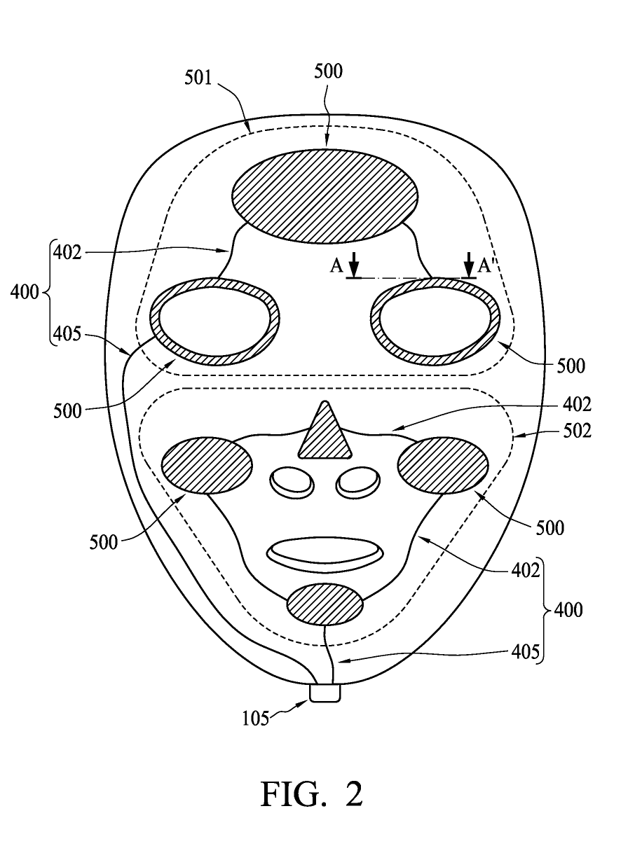

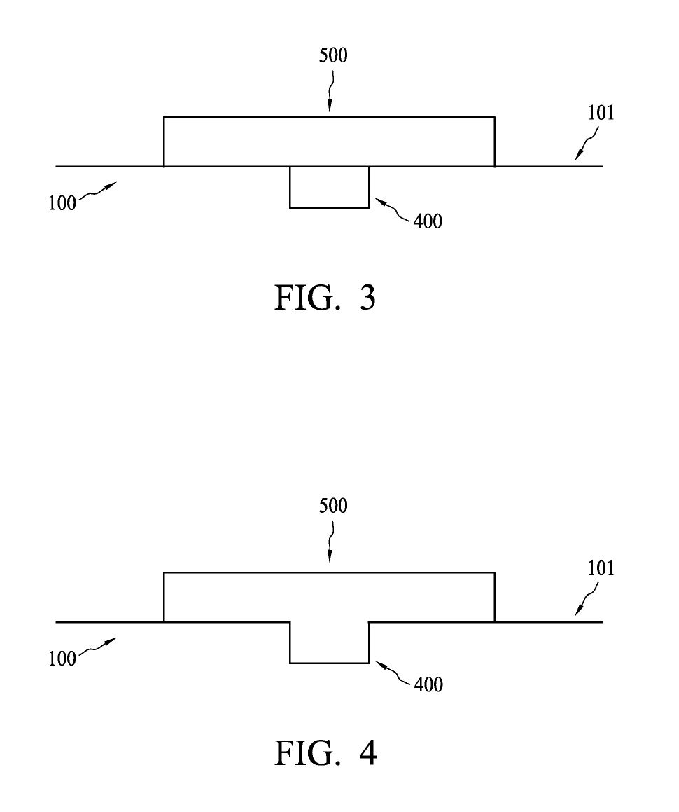

FIGS. 1 and 2 illustrate an iontophoretic transdermal device 10 according to one embodiment of the present disclosure. The iontophoretic transdermal device 10 contains an applicator 100, an electric pathway 400, and at least one electrode 500. The applicator 100 is preferably shaped as an orthomorphic projection of the covered skin. Orthomorphic projection described herein means the applicator 100 is undulated to preserve the shape of the covered skin area. For example, if the covered skin area is a face, then the applicator 100 is produced in accordance with the topography of the covered facial features (as shown in FIG. 1) such as mouth, nose, etc. Rubber-like material is preferably selected for manufacturing the applicator 100. In another preferred embodiment according to the present invention, the rubber-like material can be silicone, polydimethylsiloxane, or poly urethane. The material used for the applicator 100 also possesses high electrical resistivity. The applicator 100 with high flexibility is preferred in the present invention in order to assure the applicator 100 includes the orthomorphic projection of the covered skin area. The applicator 100 has a concave and/or convex first surface 101 which is configured to be toward cover the skin area and opposite to a second surface 102. The first surface 101 contacts the covered skin area directly, or contacts the covered skin area with the iontophoretic transdermal active agent in between. The electric pathway 400 is on the first surface as shown in FIG. 2. The word “on” in the present disclosure can mean either “disposed on”, “embedded in” or “substantially close to” the first surface 101. The term “substantially close to” means that the electric pathway 400 almost touches the first surface 101 but there might be still a small gap under 1 cm between the electric pathway 400 and the first surface 101 due to minor deviation during fabrication. The electric pathway 400 is configured to conduct electric current and is preferably made with electrical conductive material. The material used to manufacture the electric pathway 400 may be, but is not limited to, copper, silver, gold, graphite, carbon black, carbon nano-tube or graphene. The electric pathway 400 is also preferably made with flexible material in order to maintain the same topography as the applicator 100. More importantly, the electric pathway 400 can also change its shape with the matrix, i.e. the applicator 100, in order to maintain its integrity with the applicator 100 during the application. In one preferred embodiment according to the present invention, the electric pathway 400 is made with the same material as the applicator 100; additionally, conductive filler are filled, printed, or compounded into the traces of the electric pathway 400. Therefore, the electric pathway 400 is able to conduct electric current with low resistivity. The conductive fillers are preferably high electric conductive material and can be, but is not limited to, copper, silver, gold or their respective alloys. The conductive fillers can also be graphite, carbon black, carbon nano-tube or graphene. The electrodes 500 are disposed on the first surface 101 and electrically connected with the electric pathway 400. The electrodes 500 herein are preferably arranged to protrude above the first surface 101. The protrusion above the first surface 101 is preferably under 5 cm. In one embodiment of the present invention, the protrusion above the first surface 101 is preferably under 4 cm. In another embodiment of the present invention, the protrusion above the first surface 101 is preferably under 3 cm. In one embodiment of the present invention, the protrusion above the first surface 101 is preferably between 1 cm˜2 cm. In one embodiment of the present invention, the protrusion above the first surface 101 is preferably between 0.005 cm˜1 cm. FIG. 3 illustrates the cross section of cut line AA′ drawn in FIG. 2. The electrodes 500 are configured to contact the treated surface skin and are made with electrically conductive material such as metal. The metal used for the conductive fillers can be, but is not limited to, copper, silver, gold or their respective alloys. The material for the electrodes can also be graphite, carbon black, carbon nano-tube or graphene. In one embodiment of the present invention, the electrodes 500 are preferably made with the same material as the electric pathway 400. In another embodiment, both the electric pathway 400 and the electrodes 500 are made with rubber-like material, such as silicone, polydimethylsiloxane, or polyurethane, and are filled, compounded or printed with the conductive fillers. The conductive filler is preferably high electric conductive material and can be, but is not limited to, copper, silver, gold or their respective alloys. The conductive fillers can also be graphite, carbon black, carbon nano-tube or graphene. Owing to the high flexibility and protruding design, the electrode 500 is able to contact the treated surface skin substantially without any gap when the applicator 100 is applied on the treated skin area. In another embodiment, the electrodes 500 are preferably integrated with the electric pathway 400 (as shown in FIG. 4) during manufacturing. In another embodiment of present invention, the electric pathway 400 can also be enclosed by the electrode 500, that is, the bottom surface of the electric pathway 400 is coplanar with the bottom surface of the electrode 500. The bottom surface herein refer to the surface contacting with the first surface 101.

The electrodes 500 can also be grouped into at least one zone according to user requirements. In the embodiment illustrated in FIG. 2, the electrodes 500 are grouped into two different zones 501 to 502 (illustrated by the dotted lines). The zone 501 includes the electrodes 500 around the forehead and the eyes. The zone 502 includes the electrodes 500 around the nose, the cheeks and the chin. Each zone has at least one electrode 500 and the electrode(s) 500 within the same zone is (are) electrically connected by an interconnection wire 402. In addition to the interconnection wire 402, the electric pathway 400 further includes at least one extension part 405 connected to a first terminal 105 at one end of the extension part 405. The first terminal 105 can be disposed on different location of the applicator 100. It should be noted that although all the extension parts 405 can be either merged at the first terminal 105 but each extension part 405 is electrically isolated from other extension parts 405. Or in another embodiment of the present invention, there are at least two first terminals 105 located on different position of the applicator 100, and each extension part 405 is electrically connected to its respective first terminal 105. With these arrangements, each extension part 405 is able to carry a different electric current density from others. In one embodiment of the present invention, the electric current density for each zone can be manipulated independently. Also, in the embodiment with a plurality first terminals 105, the extension part 405 can be routed to its shortest path to its corresponding first terminal 105, such that the electric current loading effect can be reduced.

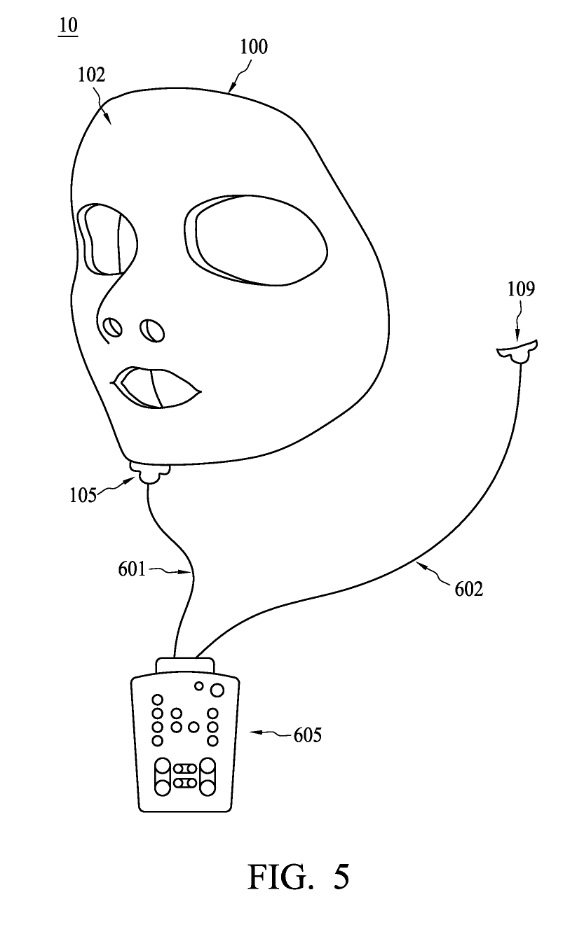

FIG. 5 illustrates another embodiment according to the present invention. An iontophoretic transdermal device 10 includes an applicator 100 and a voltage source 605. The iontophoretic transdermal device 10 further has an electric pathway 400 and at least one electrode 500 disposed on or embedded in a first surface 101 (see FIGS. 1 to 4) which is opposite to a second surface 102. The voltage source 605 can be a battery or an AC/DC converter which provides direct current (DC). The voltage source 600 further has a first pole 601 and a second pole 602. The polarity of the first pole 601 is opposite to the second pole 602. In one embodiment, the first pole 601 is a negative pole and the second pole 602 is opposite to the first pole 601, i.e., a positive pole. Each pole can preferably have a distal end to be respectively connected with a first terminal 105 and a second terminal 109.

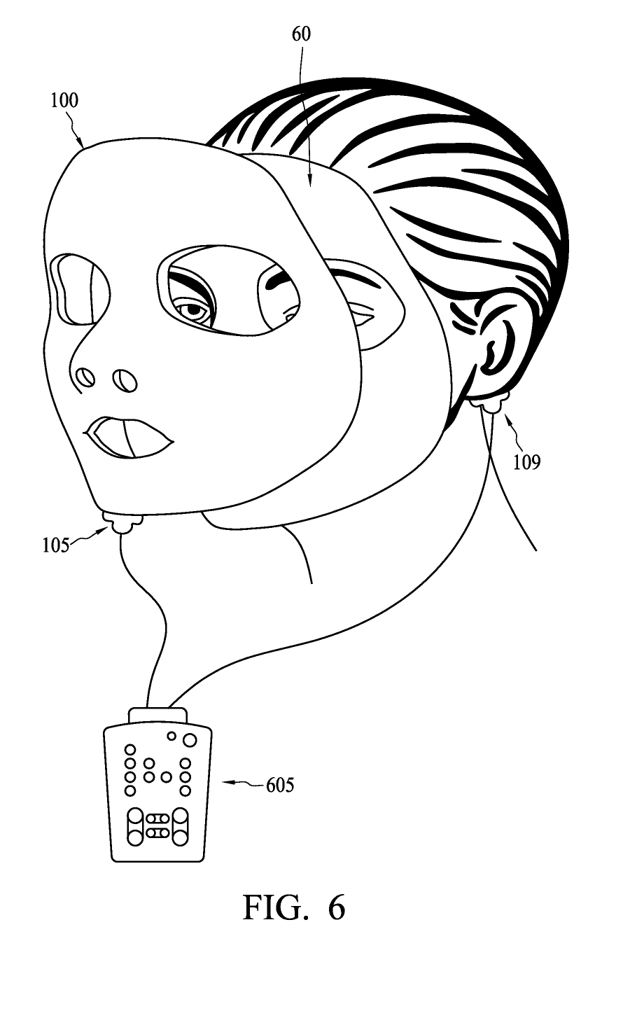

FIG. 6 illustrates the process to conduct an iontophoresis process on the skin by utilizing an embodiment according to the present invention. An active agent 60 for the iontophoresis process can be daubed on the treated facial skin, or, in another embodiment, the active agent 60 is stored in a patch configured to cover the treated skin area. Next, the applicator 100 is disposed on the active agent 60. The second terminal 109 is attached to a skin area which is different from the treated skin area, and in this embodiment is the ear. When the voltage source 605 is turned on, the voltage difference between the first terminal 105 and the second terminal 109 start inducing the ionized molecules from the active agent 60 into the treated skin. More particularly, the active agent 60 will be effectively induced into the skin at the locations where the electrodes 500 (not shown) contact the skin surface because there is substantially no gap between the electrodes 500 and the contact skin.

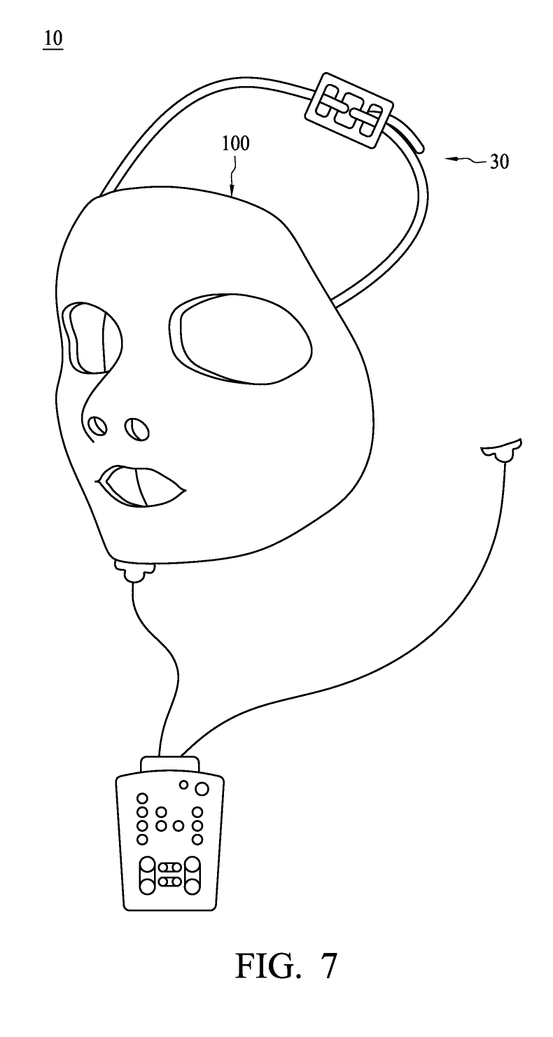

FIG. 7 illustrates another embodiment according to the present invention. The iontophoretic transdermal device 10 further includes a fastening member 30 attached to the applicator 100. In one embodiment, the fastening member 30 is attached to the rim of the applicator 100. The fastening member 30 is utilized to further secure the applicator 100 at the treated skin area. Another advantage of using the fastening member 30 is that, by affixing the fastening member 30 around the treated skin area, the applicator 100 is pressed closely to the treated skin area. Accordingly, the electrodes 500 (not shown) can be pushed more closely to the treated skin surface to assure that the gap between the electrodes 500 and the contact skin surface is substantially zero. The fastening member 30 is preferably made with a hook-and-loop tape, Velcro, a buckle belt, an adjustable rubber belt, or a strap etc.

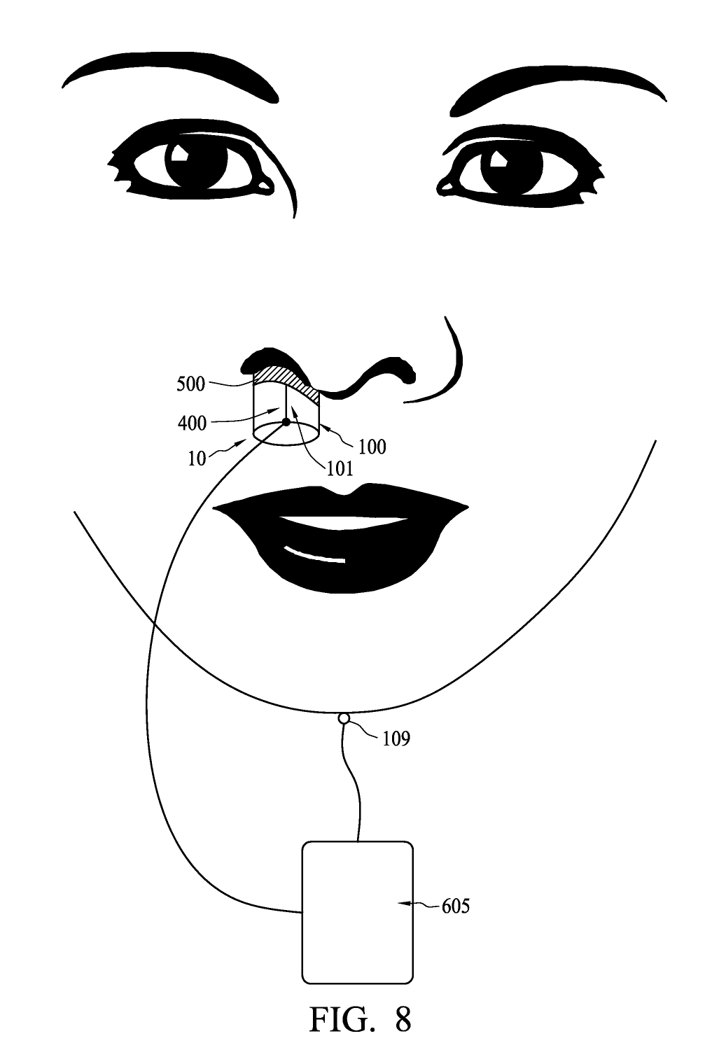

FIG. 8 illustrates another embodiment according the present invention. The applicator 100 of the iontophoretic transdermal device 10 is configured to be plugged into the nasal cavity. The applicator 100 has a first surface 101 which is convex. The transdermal device 10 has at least one electrode 500 and at least one electric pathway 400 disposed on the first surface 101. A first terminal 105 is disposed at one end of the applicator 100 and a second terminal 109 is attached to a skin surface away from the treated skin area. In the present embodiment, the second terminal 109 can be attached to the chin in order to prevent the short circuit between the first terminal 105 and the second terminal 109. The skin surface of the nasal cavity can be covered with of the active agent and the active agent is induced underneath the skin surface through the electrodes 500.

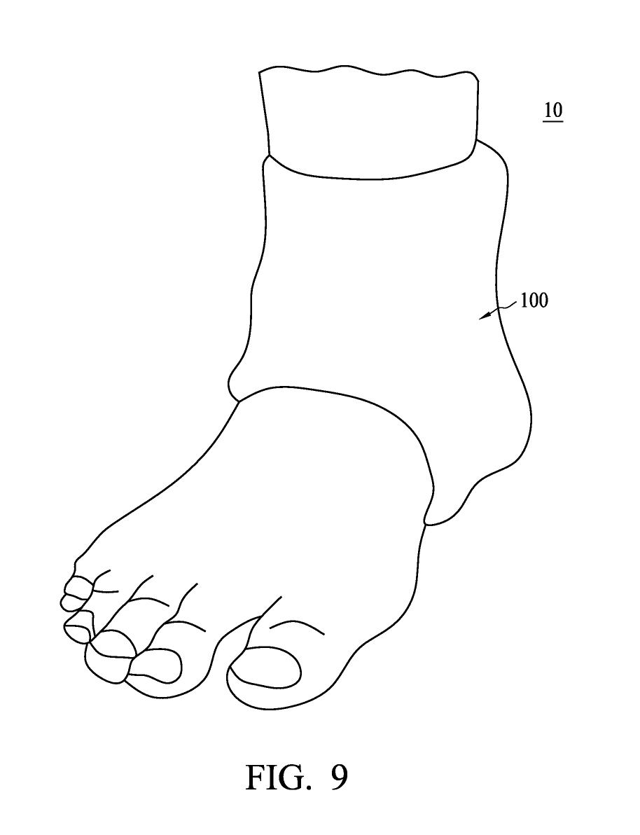

FIG. 9 illustrates another embodiment according to the present invention. In the present embodiment, the applicator 100 of the iontophoretic transdermal device 10 is configured to be applied to the skin area around the ankle.

According to the present invention, the iontophoretic transdermal device 10 can be customized into various shapes in order to fit any skin area to be treated, such as a face, nasal cavity, ankle, joints, finger, toe or any part of the skin on an object. The object is exemplified herein as a human body, but may also include an animal or any organism.

While the invention has been described above with references to specific embodiments thereof, it can be seen that many changes, modifications and variations in the materials, arrangements of parts and steps can be made without departing from the inventive concept disclosed herein. Accordingly, the spirit and broad scope of the appended claims are intended to embrace all such changes, modifications and variations that may occur to one having skill in the art upon a reading of the disclosure. All patent applications, patents and other publication cited herein are incorporated by reference in their entirety.

What is claimed is:

1. An iontophoretic transdermal device, the transdermal device comprising:
an applicator comprising a first surface, wherein the applicator is shaped as an orthomorphic projection of the covered skin area and the first surface is configured to be toward the covered skin area;
an electric pathway on the first surface; and
an electrode disposed on the first surface connected to the electric pathway and protruding above the first surface such that the electrode contacts the covered skin substantially without gap therebetween when the applicator is applied on the covered skin area.

2. The transdermal device of claim 1, wherein the material of the applicator is rubber.

3. The transdermal device of claim 2, wherein the rubber material is silicone, polydimethyl siloxane, or polyurethane.

4. The transdermal device of claim 1, wherein the electric pathway comprises conductive fillers.

5. The transdermal device of claim 4, wherein the conductive fillers are graphite, carbon nano-tube, carbon black, graphene, copper, silver, gold or their respective alloys.

6. The transdermal device of claim 1, wherein the first surface is concave or/and convex.

7. The transdermal device of claim 1, wherein the device further comprises:
a first terminal connected to the applicator;
a second terminal apart from the first terminal; and
a voltage source configured to provide DC current;
wherein the voltage source comprises a first pole connected to the first terminal and a second pole connected to the second terminal, the polarity of said first pole is opposite to the second pole.

8. The transdermal device of claim 1, wherein the device further comprises a plurality of electrodes and the electric pathway further comprises a plurality of interconnection wires, the electrodes being grouped into at least one zone, and said electrodes in the same zone being electrically connected by the corresponding interconnection wire.

9. The transdermal device of claim 8, wherein the electric pathway comprises conductive fillers.

10. The transdermal device of claim 9, wherein the conductive fillers are graphite, carbon nano-tube, carbon black, graphene, copper, silver, gold, or their respective alloys.

11. The transdermal device of claim 10, wherein the device further comprises:
a first terminal connected to the applicator;
a second terminal apart from the first terminal; and
a voltage source configured to provide DC current;
wherein the voltage source comprises a first pole connected to the first terminal and a second pole connected to the second terminal, the polarity of said first pole is opposite to the second pole.

12. The transdermal device of claim 11, wherein the electric pathway further comprises at least one extension part corresponding to each zone and said extension part is connected to the first terminal.

13. The transdermal device of claim 12, wherein each extension part is electrically isolated from other extension parts.

14. The transdermal device of claim 13, wherein the voltage source comprises a plurality of channels, and each channel controls the electric current density of its corresponding extension part independently.

15. The transdermal device of claim 1, further comprising a fastening member.

16. The transdermal device of claim 15, wherein the fastening member is a hook-and-loop tape, buckle belt, adjustable rubber belt.

17. A method of conducting a iontophoresis process, comprising:
applying iontophoresis active agent on the treated skin surface;
placing a first surface of an applicator on the treated skin surface;
contacting the skin surface with electrodes, wherein the electrodes are electrically connected to an electric pathway and the electric pathway is electrically connected to a first terminal;
connecting the first terminal to a first pole of a voltage source;
applying a second terminal on a skin area different from the treated skin surface wherein the second terminal is electrically connected to a second pole of the voltage source;
introducing DC current from the voltage source into the electric pathway and the electrode; and
inducing the active agent underneath the treated skin surface.

18. The method of claim 17, further comprising contacting the treated skin surface with a plurality of electrodes of the applicator, wherein the electrodes are grouped into at least two different zones.

19. The method according of claim 18, further comprising adjusting electric current for each zone independently.
